(19) United States
(12) Patent Application Publication
(10) Pub. No.: US 2010/0069986 A1
(43) Pub. Date: Mar. 18, 2010

(54) SYSTEMS AND METHODS FOR INCREASING PACING OUTPUT AFTER EXTERNAL HIGH-ENERGY ELECTRICAL SHOCK

(75) Inventors: Wyatt Keith Stahl, Little Canada, MN (US); Kevin J. Kindel, Bismarck, ND (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC, St. Paul, MN (US)

(21) Appl. No.: 12/539,228

(22) Filed: Aug. 11, 2009

Related U.S. Application Data
(60) Provisional application No. 61/088,937, filed on Aug. 14, 2008.

(51) Int. Cl. A61N 1/362

(52) U.S. Cl. 607/11

(57) ABSTRACT

Embodiments of the invention are related to implantable medical devices and methods for increasing pacing output after an external electrical shock, amongst other things. In an embodiment, the invention includes a medical device including a shock detection circuit; and a pacing output circuit in communication with the shock detection circuit. The pacing output circuit can be configured to generate pacing pulses. The pacing output circuit can be configured to increase the amplitude of the pacing pulses and/or increase the pulse width of the pacing pulses in response to the shock detection circuit detecting a defibrillation or cardioversion shock delivered by an external device. Other embodiments are also included herein.

This application claims the benefit of U.S. Provisional Application No. 61/088,937, filed Aug. 14, 2008, the contents of which are herein incorporated by reference.

### TECHNICAL FIELD

This disclosure relates generally to implantable medical devices, and more particularly, to implantable medical devices and methods for increasing pacing output after an external electrical shock, amongst other things.

### BACKGROUND OF THE INVENTION

Disturbances to normal sinus cardiac rhythm can pose threats to a patient's health. For example, atrial fibrillation and some types of tachycardia can result in significantly reduced cardiac output that in turn can lead to a cascade of adverse consequences. As such, medical professionals generally seek to treat many types of cardiac rhythm disturbances as quickly as possible.

In some cases, an external device may be used to deliver a high-energy defibrillation or cardioversion shock to a patient's heart in order to terminate an aberrant heart rhythm. Such shocks are frequently successful at terminating atrial tachycardias and ventricular tachycardias. Administering an external defibrillation or cardioversion shock can involve placing paddles (electrodes) on the patient's chest and initiating the discharge of an electrical pulse of energy that can be as much as 60 Amps at a voltage of 5000 Volts.

### SUMMARY OF THE INVENTION

Embodiments of the invention are related to implantable medical devices and methods for increasing pacing output after an external electrical shock, amongst other things. In an embodiment, the invention includes a medical device including a shock detection circuit; and a pacing output circuit in communication with the shock detection circuit. The pacing output circuit can be configured to generate pacing pulses. The pacing output circuit can be configured to increase the amplitude of the pacing pulses and/or increase the pulse width of the pacing pulses in response to the shock detection circuit detecting a defibrillation or cardioversion shock delivered by an external device.

In an embodiment, the invention includes a method of operating an implantable medical device. The method can include administering pacing pulses at a baseline amplitude and pulse width to the patient with the implanted medical device, monitoring electrical activity to detect external defibrillation or cardioversion pulses, and increasing the amplitude and/or pulse width of the pacing pulses, and in some embodiments the pacing rate, if an external defibrillation or cardioversion pulse is detected.

In an embodiment, the invention includes a method of making an implantable medical device. The method can include providing a shock detection circuit in electrical communication with a pacing output circuit. The pacing output circuit can be configured to generate pacing pulses and increase the amplitude and/or pulse width of the pacing pulses in response to the shock detection circuit detecting a defibrillation or cardioversion shock delivered by an external device.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is a schematic view of a patient with an implantable medical device in accordance with an embodiment of the invention.

FIG. 2 is a graph showing a train of pacing pulses over time in accordance with an embodiment of the invention.

FIG. 3 is a flowchart of a method in accordance with an embodiment of the invention.

FIG. 4 is a graph showing a train of pacing pulses over time in accordance with an embodiment of the invention.

FIG. 5 is a graph showing a train of pacing pulses over time in accordance with an embodiment of the invention.

FIG. 6 is a flowchart of a method in accordance with an embodiment of the invention.

FIG. 7 is a schematic diagram of components of a medical device in accordance with an embodiment herein.

FIG. 8 is a schematic diagram of a shock detection circuit in accordance with an embodiment herein.

FIG. 9 is a schematic diagram of components of an implantable medical device system in accordance with various embodiments herein.

FIG. 10 is a schematic diagram of components of an implantable medical device system in accordance with various embodiments herein.

FIG. 11 is a flowchart of a method in accordance with another embodiment of the invention.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

In some cases, an external device may be used to deliver a high-energy defibrillation or cardioversion shock to a patient's heart in order to terminate an aberrant heart rhythm. As used herein, the term “shock pulse” shall include both defibrillation and cardioversion shocks.

Unfortunately, administration of a shock pulse may cause the patient's pacing capture threshold to temporarily increase. As such, embodiments of the invention can include a medical device that includes a shock detection circuit and a pacing output circuit in communication with the shock detection circuit. In some embodiments, the pacing output circuit can be configured to increase the amplitude and/or pulse width of the pacing pulses in response to the shock detection circuit detecting a defibrillation or cardioversion shock delivered by an external device. In this manner, embodiments herein can function to increase the likelihood that pacing pulses delivered after an external shock pulse will be sufficient to capture the patient's heart.

It is believed that in many cases the fact that a patient has received an external shock pulse suggests that the patient may have suffered from a period of time with below optimal cardiac output. For example, a patient experiencing fibrillation of the ventricles necessitating an external shock pulse will likely have experienced substantially reduced cardiac output due to the fibrillation. As such, it is believed that many patients may benefit from an increased level of cardiac output after they receive shock pulse therapy in order to terminate an arrhythmia. In various embodiments herein, the frequency of pacing pulses is increased after the detection of an external shock pulse. By increasing the frequency of pacing pulses, it is believed that cardiac output can be increased at a time when the patient can benefit from increased cardiac output. Various aspects of exemplary devices and methods will now be described in greater detail.

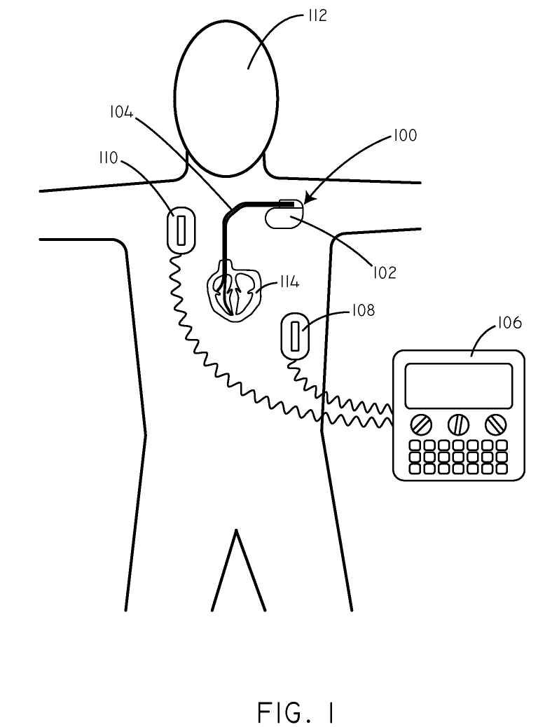

Referring now to FIG. 1, an implantable medical device system 100 is shown in accordance with an embodiment of the technology disclosed herein. The implantable medical device system 100 includes a pacemaker 102 and leads 104 including electrodes (not shown) which are arranged to provide electrical communication between the pacemaker 102 and the heart 114 of the patient 112. The pacemaker 102 generates a series of pacing pulses to stimulate contraction of the patient's heart 114. Though a pacemaker is depicted in this figure, it will be appreciated that devices in accordance with embodiments herein can include any type of implantable device with pacing functionality including, for example, implantable cardioverter-defibrillators.

In this view, an external defibrillator 106 is coupled to a pair of external electrode paddles 108 and 110. The external defibrillator 106 delivers a defibrillation or cardioversion pulse of electrical energy to the patient 112. A shock detection circuit that is part of the implantable medical device system 100 detects the defibrillation pulse and changes characteristics of the pacing pulses. By way of example, the pacemaker 102 can be configured to increase the amplitude of the pacing pulses and/or increase the pulse width of the pacing pulses in response to detecting the defibrillation pulse.

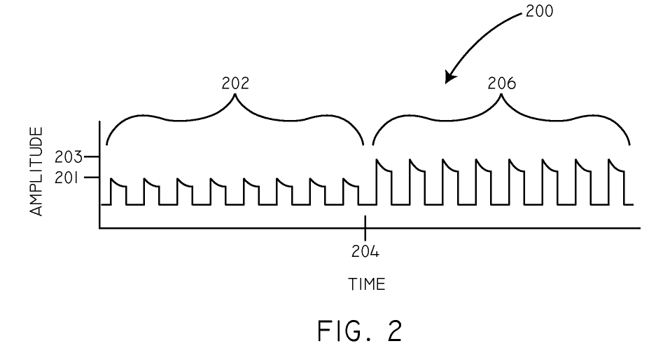

Referring now to FIG. 2, a graph is shown illustrating the amplitude of a series 200 of pacing pulses delivered to a patient over time. In a first time period 202, the pacing pulses have a baseline amplitude 201. At time 204, a defibrillation or cardioversion pulse is detected. In a second time period 206, the pacing pulses have an increased amplitude 203 over the baseline amplitude 201.

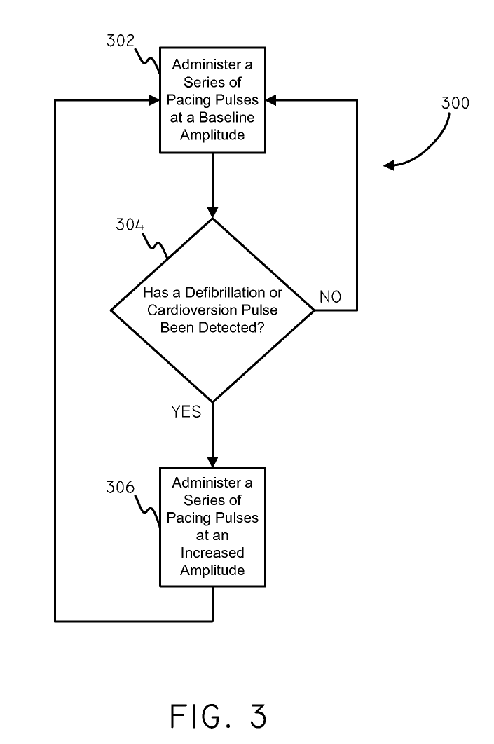

FIG. 3 shows a method is accordance with an embodiment of the invention. In a first operation 302, the system delivers a series of pacing pulses to a subject at a baseline amplitude. In some embodiments, the baseline amplitude is between about 1.5 Volts and about 3.0 Volts. In some embodiments, the baseline amplitude can be set automatically through an automated capture threshold testing procedure, including a safety margin over the minimum amplitude needed for capture. In some embodiments, the baseline amplitude can be configured to a desirable level by a clinician. The pacing pulses can be uniphasic or biphasic.

While the pacing pulses are being delivered at the baseline amplitude, the system can also monitor for shock pulses (such as defibrillation or cardioversion pulses). In a second operation 304, the system can determine whether or not an external defibrillation or cardioversion pulse has been detected. If not, then the system can simply go back to operation 302 and continue to deliver pacing pulses at the baseline amplitude. However, if a defibrillation or cardioversion pulse has been detected, the system can go to a third operation 306 and administer a series of pacing pulses at an increased amplitude.

In some embodiments, the transition between the baseline amplitude and the increased amplitude is simply a step-type change where the amplitude abruptly changes from the baseline amplitude to the increased amplitude. However, in other embodiments, the change can follow a ramp-type scheme, where the change is made gradually over a period of time.

The length of time for which the system delivers pacing pulses at the increased amplitude can vary. In some embodiments, the system can be configured to deliver pacing pulses at the increased amplitude for at least about 15 seconds. In some embodiments, the system can be configured to deliver pacing pulses at the increased amplitude for at least about 30 seconds. In some embodiments, the time period for which the system delivers pacing pulses at the increased amplitude can be configured by a clinician. In some embodiments, the system can be configured to deliver pacing pulses at an increased amplitude until the patient's cardiac rhythm has been stable for at least a specific period of time, such as for example, stable for at least 30 seconds, 1 minute, or 5 minutes.

In some embodiments, the increase in amplitude over the baseline level is at least about 1 Volt. In some embodiments the increase in amplitude over the baseline level is at least about 3 Volts. In some embodiments, the pacing pulses after detection of a shock pulse are between about 6 to about 8 Volts. In some embodiments, the increase in amplitude over the baseline level can be configured by a clinician to a desirable level.

After delivering pacing pulses at the increased amplitude, the system can return to the first operation 302 where it delivers pacing pulses at the baseline amplitude. In some embodiments, the transition between the increased amplitude and the baseline amplitude is simply a step-type change where the amplitude abruptly changes from the increased amplitude to the baseline amplitude. However, in other embodiments, the change can follow a ramp-type scheme, where the change is made gradually over a period of time.

In some embodiments, the system can be configured to increase the pulse width of pacing pulses after detection of a shock pulse. Increasing pulse width can be performed in conjunction with increasing pulse amplitude, or can be performed independently. A baseline value for pulse width of pacing pulses can be about 0.4 to about 0.5 milliseconds. In some embodiments, the pulse width can be increased to about 1 to about 2 milliseconds after detection of a shock pulse. Referring now to FIG. 4, a graph is shown illustrating the pulse width of a series 400 of pacing pulses delivered to a patient over time. In a first time period 402, the pacing pulses have a baseline pulse width 401. At time 404, a defibrillation or cardioversion pulse is detected. In a second time period 406, the pacing pulses have an increased pulse width 403 over the baseline pulse width 401.

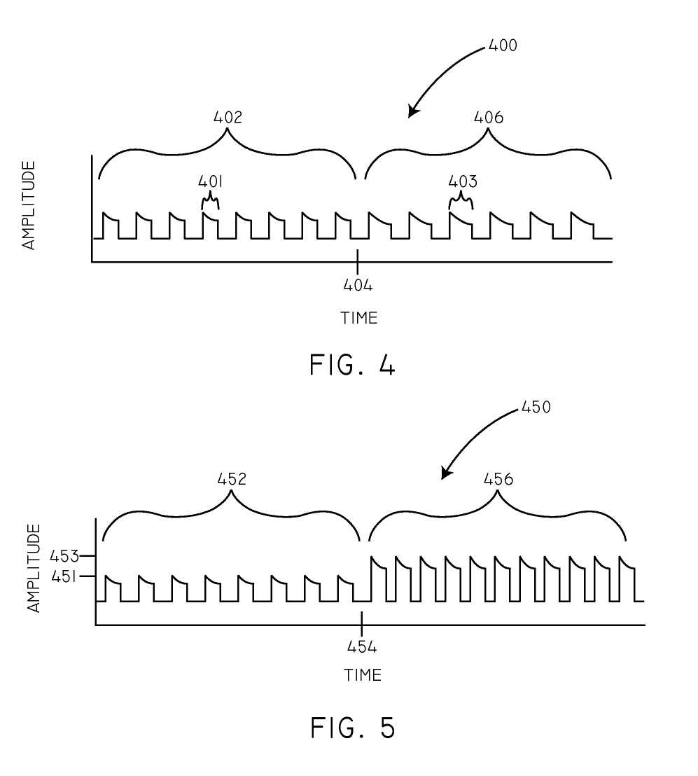

In some embodiments, the system can also be configured to increase the frequency of pacing pulses over a baseline frequency. This can be done in addition to increasing the pulse amplitude and/or pulse width. While not intending to be bound by theory, it is believed that increasing the frequency after detection of a shock pulse can be advantageous because it can lead to increased cardiac output, which may be necessary if the patient was previously in a state that necessitated administration of an external shock pulse. Referring now to FIG. 5, a graph is shown illustrating a series 450 of pacing pulses delivered to a patient over time. In a first time period 452, the pacing pulses have a baseline amplitude 451 and baseline frequency. At time 454, a defibrillation or cardioversion pulse is detected. In a second time period 456, the pacing pulses have both an increased amplitude 453 over the baseline amplitude 451 and an increased frequency over the baseline frequency.

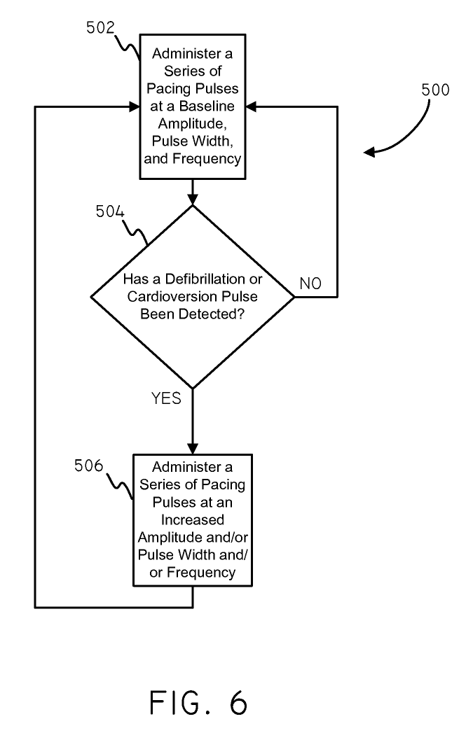

FIG. 6 shows a method 500 in accordance with an embodiment of the invention. In a first operation 502, the system delivers a series of pacing pulses to a subject at a baseline amplitude, pulse width, and frequency. The baseline amplitude, pulse width, and frequency can be configured to a desirable level by a clinician.

While the pacing pulses are being delivered at the baseline amplitude, pulse width, and frequency, the system can also monitor for shock pulses (defibrillation or cardioversion) pulses. In a second operation 504, the system can determine whether or not an external defibrillation or cardioversion pulse has been detected. If not, then the system can simply go back to the first operation 502 and continue to deliver pacing pulses at the baseline amplitude, pulse width, and frequency. However, if a defibrillation or cardioversion pulse has been detected, the system can go to a third operation 506 and administer a series of pacing pulses at an increased amplitude and/or increased pulse width, and at an increased frequency as well.

The increase in frequency over the baseline level can be configured by a clinician. In some embodiments, the increase in frequency over the baseline level is at least about 10 ppm (pulses per minute). After delivering pacing pulses at the increased amplitude and/or pulse width, and frequency, the system can return to operation 502 where it delivers pacing pulses at the baseline amplitude, pulse width, and frequency.

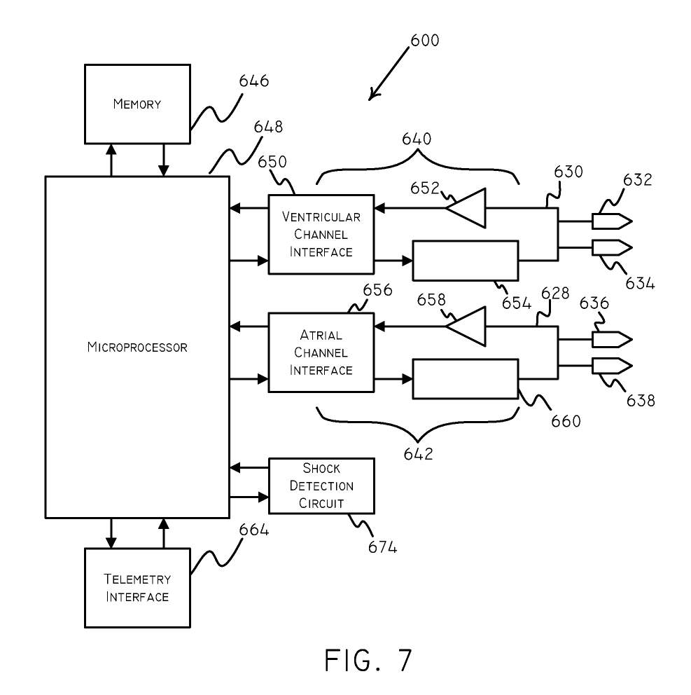

Referring now to FIG. 7, some components of an exemplary implantable system 600 in accordance with various embodiments herein are schematically illustrated. The implantable medical system 600 can include various circuitry coupled to one or more stimulation leads 630 and 628. The circuitry can include a microprocessor 648 (or processor) that communicates with a memory 646 via a bidirectional data bus. The memory 646 typically includes ROM or RAM for program storage and RAM for data storage. The system 600 can be configured to execute various operations such as processing of signals and execution of methods or operations as described herein. A telemetry interface 664 is also provided for communicating with an external unit, such as a programmer device or a patient management system.

In some embodiments, the system can include a ventricular sensing and pacing channel 640 including a first sensing amplifier 652, a first output circuit 654, and a ventricular channel interface 650 which communicates bidirectionally with a port of microprocessor 648. It will be appreciated that in some embodiments some of the elements of the system 600 shown in FIG. 7 may be omitted. For example, in some embodiments, the system may not include a ventricular pacing channel. Further, in some embodiments, additional elements may be included.

The ventricular sensing and pacing channel can be in communication with stimulation lead 630 and electrodes 632 and 634. In some embodiments, electrode 632 can be a tip electrode and electrode 634 can be a ring electrode. However, in other embodiments, the stimulation lead 630 may only include one electrode. In some embodiments, the stimulation lead 630 can include multiple electrodes.

The system can also include an atrial sensing and pacing channel 642 including second sensing amplifier 658, a second output circuit 660, and an atrial channel interface 656 which communicates bidirectionally with a port of microprocessor 648. The atrial sensing and pacing channel can be in communication with stimulation lead 628 and electrodes 636 and 638. In some embodiments, electrode 636 can be a tip electrode and electrode 638 can be a ring electrode.

For each channel, the same lead and electrodes can be used for both sensing and pacing. The channel interfaces 650 and 656 can include analog-to-digital converters for digitizing sensing signal inputs from the sensing amplifiers 652, 658 and registers which can be written to by the microprocessor 648 in order to output pulses, change the pacing pulse amplitude, and adjust the gain and threshold values for the sensing amplifiers.

A shock detection circuit 674 can also be interfaced to the microprocessor 648 for detecting external shock pulses (defibrillation and/or cardioversion shocks) to the heart. The shock detection circuit 674 can be in electrical communication with electrodes 632 and 634. Further aspects of exemplary shock detection circuits are provided in greater detail below.

### Shock Detection Circuit

It will be appreciated that many different components can be used to form a shock detection circuit. That is, many different components can be put together in various configurations in order to detect the flow of a current having a voltage exceeding a threshold amount. In general, administration of an external shock pulse (such as a defibrillation or cardioversion pulse) is expected to be of a sufficient magnitude that the electrical field it generates will interface with electrodes of an implantable medical device system that are positioned within or near the heart. Such an electrical field would be expected to generate a current within conductors (such as conductors within an electrical stimulation lead) that are in electrical communication with the electrodes.

Various components in electrical communication with the conductors can be configured in order to detect such electrical activity. By way of example, the flow of a high voltage current can be detected with a parasitic diode within a microcircuit, an inductive pick-up, or a resistive load.

Some implantable medical devices include transient voltage suppression circuits in order to limit potential damage to the device which may be caused by exposure to high voltage shocks. In accordance with some embodiments herein, a transient voltage suppression circuit can be part of a shock detection circuit.

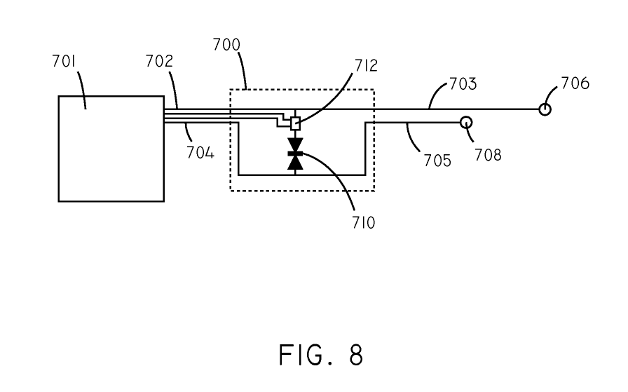

Referring now to FIG. 8, a schematic view is shown of portions of a shock detection circuit 700 including a transient voltage suppression circuit 710. Operational circuitry 702, including a pacing output circuit and/or components illustrated in FIG. 7, is electrically coupled to a first conductor 702 and a second conductor 704. The first conductor 702 and the second conductor 704 interface with the shock detection circuit 700.

The shock detection circuit 700 can include a transient voltage suppression circuit 710 and a current detector 712. In an embodiment, the transient voltage suppression circuit 710 can include two mutually opposing avalanche diodes. However, it will be appreciated that the transient voltage suppression circuit 710 can also include other components in order to prevent the flow of high voltage current into the operational circuitry 702. For example, the voltage suppression circuit 710 could also include zener diodes, a thyristor surge protection device, gas discharge tubes, metal oxide varistor, and the like. The current detector 712 can include, for example, an inductive pick-up or a resistive load. However, it will be appreciated that many different types of components and current detection circuits can be used.

The shock detection circuit 700 is electrically coupled to a first electrode 706 through a third conductor 703 and a second electrode 708 through a fourth conductor 705. In operation, when a high-voltage external pulse is delivered to a patient who has the system implanted, current is generated in the conductors 703 and 705 coupled to the first electrode 706 and the second electrode 708. However, the transient voltage circuit 710 closes or becomes a low current pathway causing the circuit to short before reaching the operational circuitry 701. As such, the high voltage current passes through the first electrode 706, the third conductor 703, the current detector 712, the voltage suppression circuit 710, the fourth conductor 705, and the second electrode 708. In the process, the current detector 712 registers that a high voltage shock has been administered, allowing the operational circuitry 701 to change the pacing pulse in response.

The shock detection circuit 700 can be disposed within many different places of an implantable system. By way of example, in some embodiments, the shock detection circuit 700 can be disposed within the housing of an implantable medical device, such as within the pulse generator can. In some embodiments, the shock detection circuit 700 can be disposed within a header attached to a pulse generator can. In still other embodiments, the shock detection circuit 700 can be disposed within stimulation leads attached to a header. In still other embodiments, the components of a shock detection circuit 700 may be split-up and disposed within different parts of the implantable system.

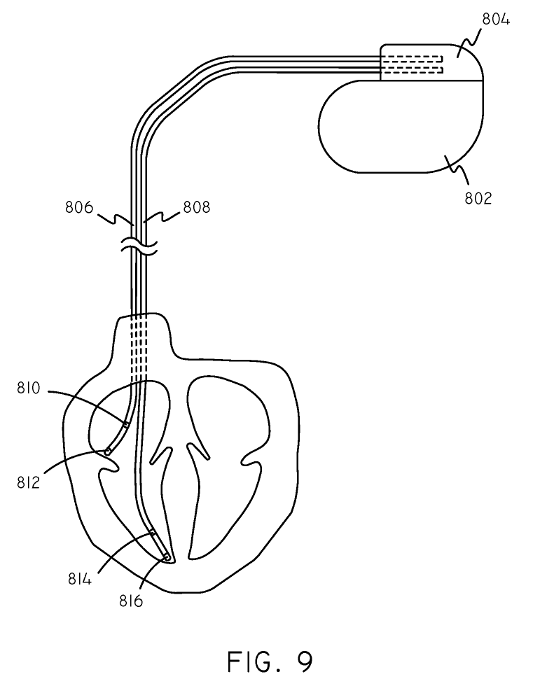

Referring now to FIG. 9, a schematic diagram of components of an implantable medical device system is shown in accordance with various embodiments herein. The system can include a pulse generator housing 802 or “can” that is coupled to a header 804. The system can also include one or more stimulation leads 806 and 808. The stimulation leads can include electrodes such as 810, 812, 814, and 816. As described above, a shock detection circuit can be disposed within the pulse generator housing 802, the header 804, and/or the stimulation leads 806 and 808.

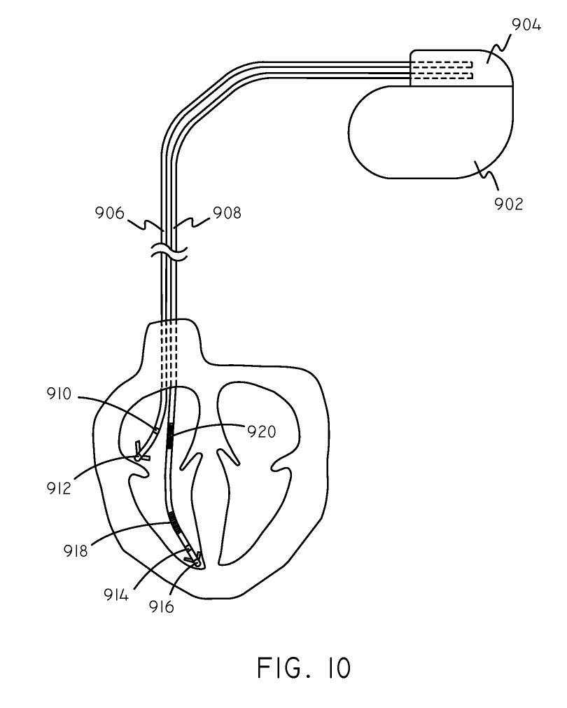

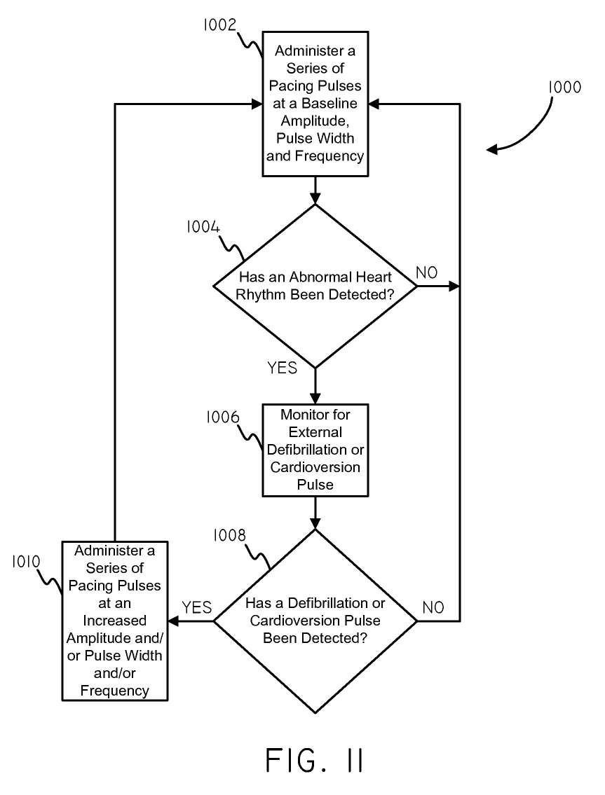

The leads of FIG. 9 are depicted as bipolar pacing/sensing leads. However, it will be appreciated that embodiments as described herein can be used in conjunction with systems having other types of leads. By way of example referring now to FIG. 10, a schematic diagram of is shown of a system including a lead with having shocking coils (electrodes). The system can include a pulse generator housing 902 that is coupled to a header 904. The system can also include stimulation leads 906 and 908. The first stimulation lead 906 can include a tip electrode 912 and a ring electrode 910. Tip electrode 912 and ring electrode 910 can be used for pacing and/or sensing. Similarly, the second stimulation lead 910 can include a tip electrode 916 and a ring electrode 914. Tip electrode 916 and ring electrode 914 can be used for pacing and/or sensing. The second stimulation lead 908 can also include a distal shocking coil 918 and a proximal shocking coil 920. As described above, a shock detection circuit can be disposed within the pulse generator housing 902, the header 904, and/or the stimulation leads 906 and 908.

In some embodiments, the system can sense whether or not a patient is experiencing an abnormal heart rhythm and then use this information with regard to detection of an external defibrillation or cardioversion pulse. For example, it is believed that in most circumstances a patient who receives an external defibrillation or cardioversion shock will have exhibited an abnormal heart rhythm in the moments leading up to administration of the external shock. This fact can be used in order to more accurately detect the administration of an external shock. In some embodiments the system can start monitoring for an external defibrillation or cardioversion shock after the system determines that a patient is experiencing an abnormal heart rhythm. In other embodiments, the system can be configured to be more sensitive to the detection of external defibrillation or cardioversion shock in response to determining that a patient is experiencing an abnormal hearth rhythm.

Referring now to FIG. 11, a flowchart of a method 1000 in accordance with an embodiment is shown. In a first operation 1002, the system delivers a series of pacing pulses to a subject at a baseline amplitude, pulse width, and frequency. In a second operation 1004, the system determines whether or not an abnormal heart rhythm has been detected. Abnormal heart rhythms can include, but are not limited to, atrial tachycardias and/or ventricular tachycardia. Such abnormal heart rhythms can be detected through algorithmic analysis of electrogram data.

If an abnormal heart rhythm is detected, then in a third operation 1006, the system can monitor for shock pulses (defibrillation or cardioversion) pulses. In a fourth operation 1008, the system can determine whether or not an external defibrillation or cardioversion pulse has been detected. If not, then the system can simply go back to the first operation 1002 and continue to deliver pacing pulses at the baseline amplitude, pulse width, and frequency. However, if a defibrillation or cardioversion pulse has been detected, the system can go to a fifth operation 1010 and administer a series of pacing pulses at an increased amplitude and/or pulse width, and/or frequency.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

One of ordinary skill in the art will understand that the modules, circuitry, and methods shown and described herein with regard to various embodiments of the invention can be implemented using software, hardware, and combinations of software and hardware. As such, the illustrated and/or described modules and circuitry are intended to encompass software implementations, hardware implementations, and software and hardware implementations.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A medical device comprising:
a shock detection circuit; and
a pacing output circuit in communication with the shock detection circuit, the pacing output circuit configured to generate pacing pulses, the pacing output circuit configured to increase the amplitude of the pacing pulses and/or increase the pulse width of the pacing pulses in response to the shock detection circuit detecting a defibrillation or cardioversion shock delivered by an external device.

2. The medical device of claim 1, the shock detection circuit comprising an inductive pick-up.

3. The medical device of claim 1, the shock detection circuit comprising a parasitic diode.

4. The medical device of claim 1, the shock detection circuit comprising a resistive load.

5. The medical device of claim 1, the shock detection circuit comprising a transient voltage suppression circuit.

6. The medical device of claim 5, the transient voltage suppression circuit comprising two mutually opposing avalanche diodes.

7. The medical device of claim 1, the pacing output circuit configured to increase the frequency of the pacing pulses in response to the shock detection circuit detecting a defibrillation or cardioversion shock delivered by an external device.

8. The medical device of claim 1, the shock detection circuit configured to detect an electrical pulse exceeding 10 Amps and 500 Volts.

9. The medical device of claim 1, the pacing output circuit configured to increase the amplitude of the pacing pulses by at least about 1 Volt.

10. The medical device of claim 1, the pacing output circuit configured to increase the amplitude of the pacing pulses by at least about 3 Volts.

11. The medical device of claim 1, the pacing output circuit configured to increase the amplitude of the pacing pulses in response to the shock detection circuit detecting a defibrillation or cardioversion shock for a period of time exceeding 30 seconds.

12. The medical device of claim 1, the pacing output circuit configured to increase the pulse width of the pacing pulses to at least about 1 millisecond in response to the shock detection circuit detecting a defibrillation or cardioversion shock for a period of time exceeding 30 seconds.

13. The medical device of claim 1, the medical device comprising a pacemaker.

14. A method of operating an implantable medical device comprising:
administering pacing pulses at a baseline amplitude and baseline pulse width to the patient with the implanted medical device;
monitoring electrical activity to detect external defibrillation or cardioversion pulses; and
increasing the amplitude and/or pulse width of the pacing pulses if an external defibrillation or cardioversion pulse is detected.

15. The method of claim 14, wherein monitoring electrical activity to detect external defibrillation or cardioversion pulses comprises detecting an electrical pulse exceeding 10 Amps and 500 Volts.

16. The method of claim 14, wherein the amplitude of pacing pulses is increased by at least 1 Volt if an external defibrillation or cardioversion pulse is detected.

17. The method of claim 14, wherein the amplitude of pacing pulses is increased by at least 3 Volts if an external defibrillation or cardioversion pulse is detected.

18. The method of claim 14, further comprising increasing the frequency of the pacing pulses if an external defibrillation or cardioversion pulse is detected.

19. A method of making an implantable medical device comprising:
providing a shock detection circuit in electrical communication with a pacing output circuit, the pacing output circuit configured to generate pacing pulses, the pacing output circuit configured to increase the amplitude of the pacing pulses and/or increase the pulse width of the pacing pulses in response to the shock detection circuit detecting a defibrillation or cardioversion shock delivered by an external device.

20. The method of claim 19, the shock detection circuit comprising a transient voltage suppression circuit.
